(19) United States
(12) Patent Application Publication
(10) Pub. No.: US 2025/0205498 A1
(43) Pub. Date: Jun. 26, 2025

(54) DETECTION OF VENTRICULAR CAPTURE FROM ATRIAL PACING BY IMPLANTABLE DEVICE

(71) Applicant: Medtronic, Inc., Minneapolis, MN (US)

(72) Inventors: Yanina Grinberg, Plymouth, MN (US); Eric R. Williams, Maple Grove, MN (US); Mackenzie E. Hall, Pittsburgh, PA (US)

(21) Appl. No.: 18/848,714

(22) PCT Filed: Mar. 27, 2023
(86) PCT No.: PCT/IB2023/053005

§ 371 Date: Sep. 19, 2024

Related U.S. Application Data
(60) Provisional application No. 63/362,019, filed on Mar. 28, 2022.

(51) Int. Cl. A61N 1/368; A61N 1/05; A61N 1/37

(52) U.S. Cl. CPC A61N 1/3688; A61N 1/056; A61N 1/371

(57) ABSTRACT

In some examples, a device comprises processing circuitry configured to identify, based on sensed electrical activity of a second chamber, a capture of the tissue of the second chamber in response to delivery of cardiac pacing to the first chamber and adjust the delivery of cardiac pacing to the first chamber in response to the identification of the capture of the second chamber.

### TECHNICAL FIELD

The disclosure relates to medical devices, and more particularly to medical devices that deliver cardiac pacing.

### BACKGROUND

Various types of implantable medical devices (IMDs) have been implanted for treating or monitoring one or more conditions of a patient. Such IMDs may be adapted to monitor or treat conditions relating to heart, muscle, nerve, brain, stomach, endocrine organs, or other organs and their related functions. Such IMDs may be associated with leads that position electrodes at a desired location, or may be leadless with electrodes integrated with and/or attached to the device housing. These IMDs may have the ability to wirelessly transmit data either to another device implanted in the patient or to another instrument located externally of the patient, or both.

A cardiac pacemaker is a medical device, e.g., an IMD, configured to deliver cardiac pacing therapy to restore a more normal heart rhythm. Cardiac pacemakers sense the electrical activity of the heart, and deliver cardiac pacing based on the sensed electrical activity, via electrodes. Some cardiac pacemakers are implanted a distance from the heart, and couple to one or more leads that intravascularly extend into the heart to position electrodes with respect to cardiac tissue. Some cardiac pacemakers are sized to be completely implanted within one of the chambers of the heart, and may include electrodes integrated with or attached to the device housing rather than leads. Some cardiac pacemakers provide dual chamber functionality, by sensing and/or stimulating the activity of both atria and ventricles, or other multi-chamber functionality. A cardiac pacemaker may provide multi-chamber functionality via leads that extend to respective heart chambers, multiple cardiac pacemakers may provide multi-chamber functionality by being implanted in respective chambers, or a cardiac pacemaker implanted in one chamber may sense and pace multiple chambers.

### SUMMARY

In general, this disclosure is directed to operation of devices implanted within a single chamber of the heart and directed to deliver cardiac pacing to more than one chamber of the heart. More particularly, this disclosure is directed to techniques that may be implemented by such devices to detect capture of ventricular tissue in response to cardiac pacing delivered to atrial tissue and responsively adjust cardiac therapy.

In one example, this disclosure may be directed to a device comprising a housing; a first electrode and a second electrode, wherein the first electrode is configured to contact tissue of a first chamber of the heart, and wherein the second electrode is configured to penetrate into tissue of a second chamber of the heart that is separate from the first chamber of the heart; sensing circuitry within the housing, wherein the sensing circuitry is configured to sense electrical activity of the first chamber via the first electrode and electrical activity of the second chamber via the second electrode; therapy delivery circuitry within the housing, wherein the therapy delivery circuitry is configured to deliver cardiac pacing to the first chamber via the first electrode and the second chamber via the second electrode; and processing circuitry within the housing. The processing circuitry is configured to identify, based on the sensed electrical activity of the second chamber, a capture of the tissue of the second chamber in response to delivery of cardiac pacing to the first chamber by the therapy delivery circuitry via the first electrode; and adjust the cardiac pacing to the first chamber based on the identification of the capture of the second chamber.

In another example, this disclosure may be directed to a method comprising delivering cardiac pacing from a device to a first chamber of a heart via a first electrode of the device; sensing, by the device and via a second electrode of the device that penetrates into tissue of a second chamber of the heart that is separate from the first chamber of the heart, electrical activity of the second chamber; identifying, by the device and based on the sensed electrical activity of the second chamber, a capture of the tissue of the second chamber in response to delivery of cardiac pacing to the first chamber via the first electrode; and adjusting, by the device, the cardiac pacing to the first chamber by the therapy delivery circuitry in response to the identification of the capture of the second chamber.

In another example, this disclosure may be directed to a method comprising delivering test cardiac pacing pulses from a device to a first chamber of a heart via a first electrode of the device; detecting, by the device and via the first electrode and a second electrode of the device that penetrates into tissue of a second chamber of the heart that is separate from the first chamber of the heart, electrical activity of the first chamber and the second chamber, respectively; determining, by the device and based on the sensed electrical activity of the first chamber, a capture threshold of the tissue of the first chamber; determining, by the device and based on the sensed electrical activity of the second chamber, a cross-chamber capture threshold of the tissue of the second chamber and a maximum pacing output for the first chamber; and based on a comparison of the capture threshold and the maximum pacing output, communicating, by the device to another device via a network, an indication to adjust a position of the first electrode within the first chamber.

In another example, this disclosure may be directed to a computer readable storage medium comprising instructions that, when executed, cause processing circuitry within a device to perform the method described above.

This summary is intended to provide an overview of the subject matter described in this disclosure. It is not intended to provide an exclusive or exhaustive explanation of the methods and systems described in detail within the accompanying drawings and description below.

### BRIEF DESCRIPTION OF THE DRAWINGS

The details of one or more examples of this disclosure are set forth in the accompanying drawings and the description below. Other features, objects, and advantages of this disclosure will be apparent from the description and drawings, and from the claims. Reference is made to the attached drawings, wherein elements have the same reference numeral designations represent similar elements throughout.

FIG. 1 is a conceptual drawing illustrating an example device implanted in the heart of a patient, in accordance with one or more aspects of this disclosure.

FIG. 2 is a perspective drawing illustrating the example device of FIG. 1, in accordance with one or more aspects of this disclosure.

FIG. 3 is a functional block diagram illustrating an example configuration of the IMD of FIGS. 1 and 2, in accordance with one or more aspects of this disclosure.

FIG. 4 is a conceptual diagram of the device of FIGS. 1-3 implanted at a target implant site.

FIG. 5 is a block diagram illustrating an example configuration of computing device 120.

FIG. 6 is a plot diagram showing an example electrogram (EGM) graph of normal atrium and ventricle activity.

FIG. 7 is a plot diagram showing an example EGM graph of the capture of a second chamber in response to the delivery of stimulation signals to a first chamber.

FIG. 8 is a flowchart illustrating an example method for detecting the capture of a second chamber in response to the delivery of stimulation signals to a first chamber.

FIG. 9 is a flowchart illustrating an example method for confirming the detection of the capture of a second chamber in response to the delivery of stimulation signals to a first chamber.

FIG. 10 is a flowchart illustrating an example method for detecting the capture of a second chamber in response to the delivery of stimulation signals to a first chamber during the implantation of an implantable medical device.

### DETAILED DESCRIPTION

In general, this disclosure is directed to medical devices, such as implantable medical devices (IMDs) having housings sized for implantation wholly within a single chamber of the heart. More particularly, this disclosure is directed to techniques implementable by such medical devices that allow for the detection of capture of ventricular tissue in response to the application of cardiac pacing therapy to atrial tissue. In some examples, in addition to the detection of ventricular capture, the systems and methods may also allow for the adjustment of cardiac therapy in response to the detection of ventricular capture.

In some cases, an IMD implanted wholly within a first chamber of the heart (e.g., in the right atrium) may deliver a stimulation signal into the first chamber with a sufficiently high magnitude that the stimulation signal travels into the wall tissue of a second chamber of the heart (e.g., the right ventricle) and causes stimulation of the wall tissue of the second chamber (e.g., causing ventricular capture).

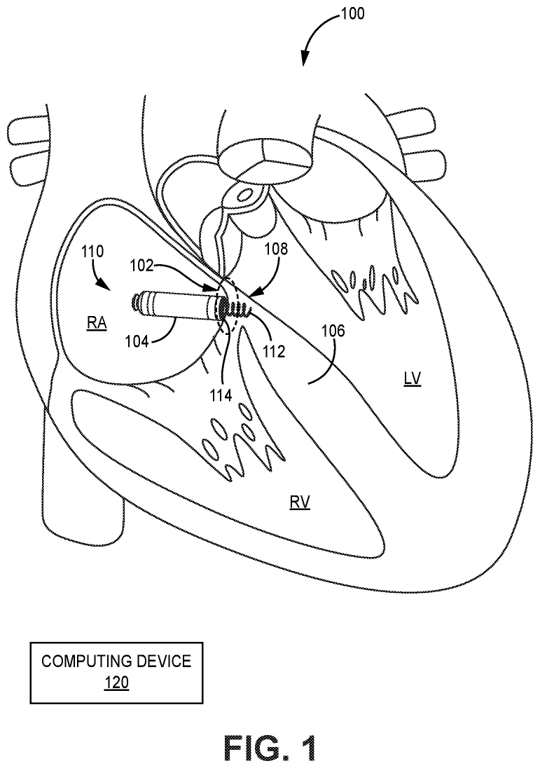

FIG. 1 is a conceptual drawing illustrating an example device 104 implanted in the heart 100 of a patient, in accordance with one or more aspects of this disclosure. Device 104 is shown implanted in the right atrium (RA) of the patient's heart 100 in a target implant region 102, such as triangle of Koch, in heart 100 of the patient with a distal end of device 104 directed towards the left ventricle (LV) of the patient's heart 100. Although in the example of FIG. 1 the distal end of device 104 is directed toward the LV, the distal end may be directed to other targets, such as interventricular septum of heart 100, in some examples. Target implant region 102 may lie between the bundle of His and the coronary sinus and may be adjacent to the tricuspid valve. In other examples, device 104 may be implanted in the left atrium (LA) with a distal end of device 104 directed towards the right ventricle (RV).

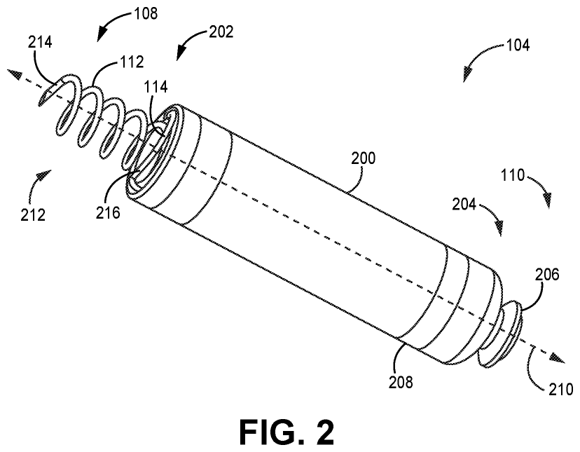

Device 104 includes a distal end 108 and a proximal end 110. Distal end 108 includes a first electrode 114 and a second electrode 112. Second electrode 112 extends from distal end 108 and may penetrate through the wall tissue of a first chamber (e.g., the RA in the illustrated example) into wall tissue of a second chamber (e.g., the LV in the illustrated example). First electrode 114 extends from distal end 108 and is configured to flexibly maintain contact with the wall tissue of the first chamber without penetration of the wall tissue of the first chamber by the first electrode 114. The configurations of electrodes 112 and 114 illustrated in FIGS. 1 and 2 are merely examples. In general, the techniques of this disclosure may be implemented by any device having one or more electrodes configured to pace and sense a ventricle, e.g., by penetration of myocardial tissue, and one or more electrodes configured to pace and sense an atrium, e.g., by contacting the tissue.

The configuration of electrodes 112 and 114 illustrated in FIG. 1 allows device 104 to sense cardiac signals and/or deliver cardiac pacing to multiple chambers of heart 100 (e.g., the RA and ventricles in the illustrated example). In this manner, the configuration of electrodes 112 and 114 may facilitate the delivery of A-V synchronous pacing by single device 104 implanted within the single chamber (e.g., the RA). While device 104 is implanted at target implant region 102 to sense in and/or pace the RA and ventricles in the example shown in FIG. 1, a device having an electrode configuration in accordance with the examples of this disclosure may be implanted at any of a variety of locations to sense in and/or pace any two or more chambers of heart 12. For example, device 104 may be implanted at region 102 or another region, and second electrode 112 may extend into tissue, e.g., myocardial tissue, of the LV or interventricular septum to, for example, facilitate the delivery of A-V synchronous pacing. Furthermore, a device having an electrode configuration in accordance with the examples of this disclosure may be implanted at any of a variety of locations within a patient for sensing and/or delivery of therapy to other patient tissue.

FIG. 1 also illustrates a computing device 120, which may be part of a medical device system with device 104. Computing device 120 may be an external device configured to wirelessly communicate with device 104, e.g., to retrieve data from device 104, including any data described herein. In some examples, computing device 120 may include a user interface to present retrieved data to a user, such as a patient or clinician. In some examples, computing device 120 may program parameters that control the operation of device 104, such as cardiac pacing parameters or sensing parameters.

FIG. 2 is a perspective drawing illustrating device 104. Device 104 includes a housing 200 that defines a hermetically sealed internal cavity. Housing 200 may be formed from a conductive material including titanium or titanium alloy, stainless steel, MP35N (a non-magnetic nickel-cobalt-chromium-molybdenum alloy), platinum alloy or other bio-compatible material or metal alloy, or other suitable conductive material. In some examples, housing 200 is formed from a non-conductive material including ceramic, glass, sapphire, silicone, polyurethane, epoxy, acetyl co-polymer plastics, polyether ether ketone (PEEK), a liquid crystal polymer, other biocompatible polymer, or other suitable non-conductive material.

Housing 200 extend from distal end 202 and proximal end 204. In some examples, housing can be cylindrical or substantially cylindrical but may be other shapes, e.g., prismatic or other geometric shapes. Housing 200 may include a delivery tool interface member 206, e.g., at proximal end 204, for engaging with a delivery tool during implantation of device 104.

All, substantially all, or a portion of housing 200 may function as an electrode 208, e.g., an anode, during pacing and/or sensing. In some examples, electrode 208 can circumscribe a portion of housing 200 at or near proximal end 204. Electrode 208 can fully or partially circumscribe housing 200. FIG. 2 shows electrode 208 extending as a singular band. Electrode 208 can also include multiple segments spaced a distance apart along a longitudinal axis 210 of housing 200 and/or around a perimeter of housing 200.

When housing 200 is formed from a conductive material, such as titanium alloy, portions of housing 200 may be electrically insulated by a non-conductive material, such as a coating of parylene, polyurethane, silicone, epoxy or other biocompatible polymer, or other suitable material. For the portions of housing 200 without the nonconductive material, one or more discrete areas of housing 200 with conductive material can be exposed to define electrode 208.

When housing 200 is formed from a non-conductive material, such as a ceramic, glass or polymer material, an electrically-conductive coating or layer, such as a titanium, platinum, stainless steel, alloys thereof, a conductive material may be applied to one or more discrete areas of housing 200 to form electrode 208.

In some examples, electrode 208 may be a component, such as a ring electrode, that is mounted or assembled onto housing 200. Electrode 208 may be electrically coupled to internal circuitry of device 104 via electrically-conductive housing 200 or an electrical conductor when housing 200 is a non-conductive material. In some examples, electrode 208 is located proximate to proximal end 204 of housing 200 and can be referred as a proximal housing-based electrode. Electrode 208 can also be located at other positions along housing 200, e.g., located proximately to distal end 202 or at other positions along longitudinal axis 210.

Each of first electrode 114 and second electrode 112 extends from a first end that is fixedly attached to housing 200 at or near distal end 202, to a second end that, in the example of FIG. 2, is not attached to housing 200 other than via the first end. First electrode 114 includes one or more coatings configured to define a first electrically active region 216 and second electrode 112 includes one or more coatings configured to define a second electrically active region 214. In some examples, second electrically active region 214 can be proximate to the second, e.g., distal, end of second electrode 112 and first electrically active region 216 is proximate to either end of first electrode 114. In the example of FIG. 2, first electrically active region 216 includes the distal end of first electrode 114.

First electrode 114 and second electrode 112 may be formed of an electrically conductive material, such as titanium, platinum, iridium, tantalum, or alloys thereof. First electrode 114 and second electrode 112 may be coated with an electrically insulating coating, e.g., a parylene, polyurethane, silicone, epoxy, or other insulating coating, to reduce the electrically conductive active surface area of first and second electrodes 114 and 112 and thereby define first and second electrically active regions 216 and 214. Defining first and second electrically active regions 216 and 214 by covering portions with an insulating coating may increase the electrical impedance of first and second electrodes 114 and 112 and thereby reduce the current delivered during a pacing pulse that captures the cardiac tissue. A lower current drain conserves the power source, e.g., one or more rechargeable or non-rechargeable batteries, of device 104.

In some examples, first and second electrodes 114 and 112 may have an electrically conducting material coating on first and second electrically active regions 216 and 214 to define the active regions. For example, first and second electrically active regions 216 and 214 may be made of substantially similar material or may be made of different material from one another.

In the example of FIG. 2, second electrode 112 takes the form of a helix. In some examples, a helix is an object having a three-dimensional shape like that of a wire wound uniformly in a single layer around a cylindrical or conical surface such that the wire would be in a straight line if the surface were unrolled into a plane.

As illustrated in FIG. 2, second electrode 112 may be a right-hand wound helix, and first electrode 114 may be a left-hand wound partial helix, although in other example the handedness of the electrodes may be switched or the electrodes may have the same handedness as each other. In the example of FIG. 2, the partial helix and helix, defined by first electrode 114 and second electrode 112, respectively, have the same pitch, although they may have different pitches in other examples. In some examples, one or both of electrodes 112 and 114 may have a shape other than helical. For example, first electrode 114 may have a loop shape. As another example, second electrode 112 configured to penetrate tissue of another chamber may be configured as one or more elongate darts, barbs, or tines.

First and second electrodes 114 and 112 can also vary in size and shape in order to enhance tissue contact of first and second electrically active regions 216 and 214. For example, first and second electrodes 114 and 112 can have a round cross section or could be made with a flatter cross section (e.g., oval or rectangular) based on tissue contact specifications. The size and shape of first and second electrodes 114 and 112 can also be determined by stiffness requirements. For example, stiffness requirements may vary based on the expected implantation requirements, including the tissue into which the first and second electrodes 114 and 112 are implanted or contact, as well as how long device 104 is intended to be implemented.

The distal end of second electrode 112 can have a conical, hemi-spherical, or slanted edge distal tip with a narrow tip diameter, e.g., less than 1 millimeter (mm), for penetrating into and through tissue layers. In some examples, the distal end of first electrode can be a sharpened or angular tip or sharpened or beveled edges, but the degree of sharpness may be constrained to avoid a cutting action that could lead to lateral displacement of the distal end of second electrode 112 and undesired tissue trauma. In some examples, second electrode 112 may have a maximum diameter at its base that interfaces with housing distal end 32. In such examples, the diameter of second electrode 112 may decrease from housing distal end 202 to the distal end of second electrode 112.

The outer dimensions of second electrode 112 can be substantially straight and cylindrical, with second electrode 112 being rigid in some examples. In some examples, first and second electrodes 114 and 112 can have flexibility in lateral directions, being non-rigid to allow some flexing with heart motion. In a relaxed state, when not subjected to any external forces, first and second electrodes 114 and 112 can be configured to maintain a distance between first and second electrically active regions 216 and 214 and housing distal end 202.

Distal end of second electrode 112 can pierce through one or more tissue layers to position second electrically active region 214 within a desired tissue layer, e.g., the ventricular myocardium or interventricular septum. Accordingly, second electrode 112 extends a distance from housing distal end 202 corresponding to the expected pacing site depth and may have a relatively high compressive strength along its longitudinal axis, which may be substantially similar to longitudinal axis 210, to resist bending in a lateral or radial direction when a longitudinal, axial, and/or rotational force is applied, e.g., to proximal end 204 of housing 200 to advance device 104 into the tissue at target implant region 102. By resisting bending in a lateral or radial direction, second electrode 112 can maintain a spacing between a plurality of windings of second electrode 112 when second electrode 112 is a helix electrode. Second electrode 112 may be longitudinally non-compressive. First electrode 114 may also be elastically deformable in lateral or radial directions when subjected to lateral or radial forces, however, to allow temporary flexing, e.g., with tissue motion, but returns to its normally straight position when lateral forces diminish. In some examples, when second electrode 112 is not exposed to any external force, or to only a force along its longitudinal axis (substantially similar to longitudinal axis 210), second electrode 112 retains a straight, linear position as shown.

In some examples, first electrode 114 or electrode 208 may be paired with second electrode 112 for sensing ventricular signals and delivering ventricular pacing pulses. In some examples, first electrode 114 may be paired with electrode 208 or second electrode 112 for sensing atrial signals and delivering pacing pulses to atrial myocardium in target implant region 102. In other words, electrode 208 may be paired, at different times, with both second electrode 112 or first electrode 114 for either ventricular or atrial functionality, respectively, in some examples. In some examples, first and second electrodes 114 and 112 may be paired with each other, with different polarities, for atrial and ventricular functionality.

In some examples, first electrode 114 may be configured as an atrial cathode electrode for delivering pacing pulses to the atrial tissue at target implant region 102 in combination with electrode 208. First electrode 114 and electrode 208 may also be used in sense atrial P-waves for use in controlling atrial pacing pulses (delivered in the absence of a sensed P-waves) and for controlling atrial-synchronized ventricular pacing pulses delivered using second electrode 112 as a cathode and electrode 208 as the return anode.

At distal end 108, device 104 includes a distal fixation assembly 212 including first electrode 114, second electrode 112, and housing distal end 202. A distal end of second electrode 112 can be configured to rest within a ventricular myocardium of the patient, and first electrode 114 can be configured to contact an atrial endocardium of the patient. In some examples, distal fixation assembly 212 can include more or less electrodes than two electrodes. In some examples, distal fixation assembly 212 may include one or more second electrodes along housing distal end 202. For example, distal fixation assembly 212 may include three electrodes, which may be substantially similar or different from one another. Spacing between a plurality of first electrode 114 may be at an equal or unequal distance. First electrode(s) 114 may be individually selectively coupled to sensing and/or pacing circuitry enclosed by housing 200 for use as an anode with second electrode 112 or as an atrial cathode electrode, or may be electrically common and not individually selectable.

First electrode 114 may be configured to flexibly maintain contact with wall tissue of the heart chamber in which device 104 is implanted, e.g., the RA endocardium, despite variations in the tissue surface or in the distance between distal end 202 of housing 200 and the tissue surface, which may occur as the wall tissue moves during the cardiac cycle.

Although described in the context of device 104, which is configured for implantation within heart 100, the techniques of this disclosure may be implemented in any device or system having first and second electrodes configured as described herein. For example, a device implanted outside the heart, e.g., pectorally or otherwise subcutaneously, may be coupled to one or more leads that extend into the heart. The distal end of the single lead may include first and second electrodes configured as described herein (e.g., electrodes 112, 114 as shown in FIG. 2), through which the device may deliver pacing and sense electrical activity of the heart as described herein. For example, the single lead may reside in a first chamber of the heart (e.g., an atrium such as the RA) with one of its electrodes in contact with tissue of the first chamber, and the other of its electrodes penetrating into tissue of a second, separate chamber of the heart (e.g., a ventricle such as the LV).

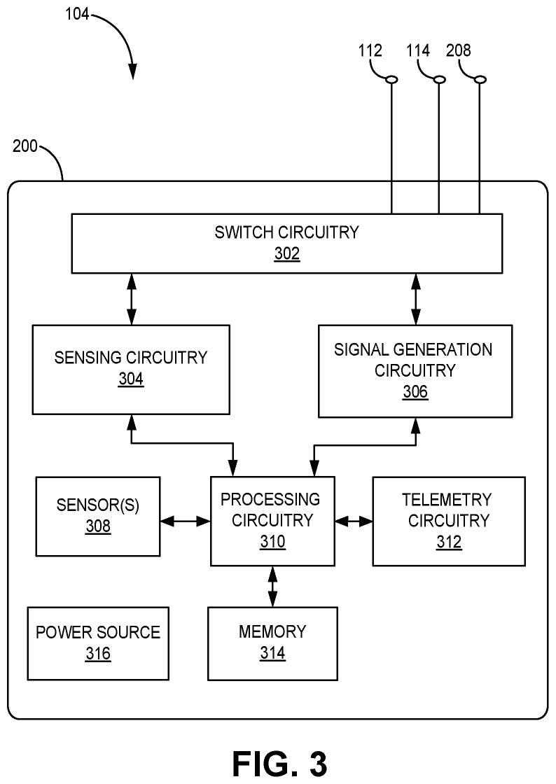

FIG. 3 is a functional block diagram illustrating an example configuration of device 104. As illustrated in FIG. 3, device 104 includes electrodes 112 and 114, which may be configured as described with respect to FIGS. 1 and 2. For example, as described with respect to FIGS. 1 and 2, second electrode 112 may be configured to extend from distal end 202 of housing 200 and may penetrate through the wall tissue of a first chamber (e.g., RA) into the wall tissue of a second chamber (e.g., LV). First electrode 114 extends from distal end 202 of housing 200 and may be configured to flexibly maintain contact with the wall tissue of the first chamber without penetration of the wall tissue of the first chamber by the first electrode.

In the example shown in FIG. 3, device 104 includes switch circuitry 302, sensing circuitry 304, signal generation circuitry 306, sensor(s) 308, processing circuitry 310, telemetry circuitry 312, memory 314, and power source 316. The various circuitry may be, or include, programmable or fixed function circuitry configured to perform the functions attributed to respective circuitry. Memory 314 may store computer-readable instructions that, when executed by processing circuitry 310, cause device 104 to perform various functions. Memory 314 may be a storage device or other non-transitory medium. The components of device 104 illustrated in FIG. 3 may be housed within housing 200.

Signal generation circuitry 306 is configured to generate stimulation signals, e.g., cardiac pacing pulses. Signal generation circuitry 306 may include, as examples, current or voltage sources, capacitors, charge pumps, or other signal generation circuitry. Switch circuitry 302 is coupled to electrodes 112, 114, and 208 and may include one or more switch arrays, one or more multiplexers, one or more switches (e.g., a switch matrix or other collection of switches), one or more transistors, or other electrical circuitry. Switch circuitry 302 is configured to direct stimulation signals from signal generation circuitry 306 to a selected combination of electrodes 112, 114, and 208, having selected polarities, e.g., to selectively deliver pacing pulses to the RA, ventricles, or interventricular septum of heart 100. For example, in order to pace one or both of the ventricles, switch circuitry 302 may couple second electrode 112, which has penetrated to wall tissue of a ventricle or the intraventricular septum, to signal generation circuitry 306 as a cathode, and one or both of first electrode 114 or electrode 208 to signal generation circuitry 306 as an anode. As another example, in order to pace the RA, switch circuitry 302 may couple first electrode 114, which flexibly maintains contact with the RA endocardium, to signal generation circuitry 306 as a cathode, and one or both of second electrode 112 or electrode 208 to signal generation circuitry 306 as an anode.

Switch circuitry 302 may also selectively couple sensing circuitry 304 to selected combinations of electrodes 112, 114, and 208, e.g., to selectively sense the electrical activity of either the atria or ventricles of heart 100. Sensing circuitry 304 may include filters, amplifiers, analog-to-digital converters, or other circuitry configured to sense cardiac electrical signals via electrodes 112, 114, and 208. For example, switch circuitry 302 may couple each of second electrode 112 and first electrode 114 (in combination with electrode 208) to respective sensing channels provided by sensing circuitry 304 to respectively sense either ventricular or atrial cardiac electrical signals. In some examples, sensing circuitry 304 is configured to detect events, e.g., depolarizations, within the cardiac electrical signals, and provide indications thereof to processing circuitry 310. In this manner, processing circuitry 310 may determine the timing of atrial and ventricular depolarizations, and control the delivery of cardiac pacing, e.g., AV synchronized cardiac pacing, based thereon.

In some examples, sensing circuitry 304 may be configured to sense and record electrical activity within a first chamber of heart 100 (e.g., the RA) and a second chamber of heart 100 (e.g., the LV). In some examples, electrical activity with the first and/or second camber may represent the capture of the corresponding chamber. For example, sensing circuitry 304 may detect electrical activity in the RA corresponding to atrial capture and may also detect electrical activity in the LV corresponding to ventricular capture.

Processing circuitry 310 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 310 herein may be embodied as firmware, hardware, software or any combination thereof.

Processing circuitry 310 may be configured to identify the presence of a capture of the tissue of a second chamber (e.g., LV) in response to delivery of cardiac pacing to a first chamber (e.g., RA). The capture of the tissue of the second chamber may also be referred to as cross-chamber capture. In some examples, processing circuitry 310 may identify the presence of capture using sensed data from sensing circuitry 304.

In some examples, processing circuitry 310 may be configured to send to signal generation circuitry 306 and/or switch circuitry 302 instructions to modify one or more stimulation signals, e.g., pacing pulses, in response to an identification of capture. Modification of the one or more stimulation signals may include adjust the pulse amplitude, pulse width, and/or frequency of each stimulation signal. In some examples, modification of the one or more stimulation signals may include a termination of the delivery of stimulation signals to the second chamber.

Sensor(s) 308 may include one or more sensing elements that transduce patient physiological activity to an electrical signal to sense values of a respective patient parameter. Sensor(s) 308 may include one or more accelerometers, optical sensors, chemical sensors, temperature sensors, pressure sensors, or any other types of sensors. Sensor(s) 308 may output patient parameter values that may be used as feedback to control sensing and delivery of therapy by device 104.

Telemetry circuitry 312 (also referred to as “communications circuitry 312”) supports wireless communication between device 104 and an external programmer or another computing device, such as computing device 120, under the control of processing circuitry 310. Processing circuitry 310 of device 104 may receive, as updates to operational parameters from computing device 120, and provide collected data, e.g., sensed heart activity or other patient parameters, to computing device 120 via telemetry circuitry 312. In some examples, processing circuitry 310 of device 104 may send, via telemetry circuitry 312, notifications to computing device 120 that processing circuitry 310 has identified the capture of the wall tissue of a second chamber of heart 100 (e.g., LV) as a result of stimulation signals delivered by device 104 to a first chamber of heart 100 (e.g., RA). Telemetry circuitry 312 may accomplish communication by radiofrequency (RF) communication techniques, e.g., via an antenna (not shown).

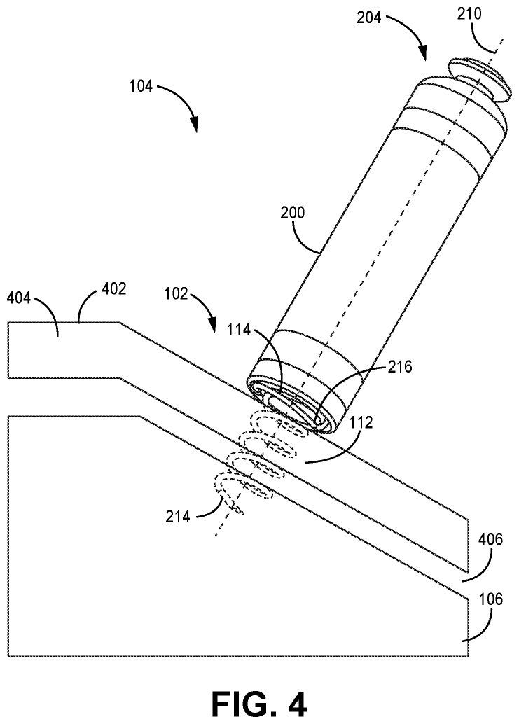

FIG. 4 is a conceptual diagram of device 104 of FIG. 1 or 2 implanted at target implant region 102. Second electrode 112 may be inserted such that tissue becomes engaged with the helix of second electrode 112. As second electrode 112 becomes engaged with tissue, second electrode 112 pierces into the tissue at target implant region 102 and advances through atrial myocardium 404 and central fibrous body 406 to position second electrically active region 214 in ventricular myocardium 106 as shown in FIG. 4. In some examples, second electrode 112 penetrates into the interventricular septum. In some examples, second electrode 112 does not perforate entirely through the ventricular endocardial or epicardial surface.

In some examples, manual pressure applied to the housing proximal end 204, e.g., via an advancement tool, provides the longitudinal force to pierce the cardiac tissue at target implant region 102. In some examples, actuation of an advancement tool rotates device 104 and second electrode 112 configured as a helix about longitudinal axis 210. The rotation of the helix about the longitudinal axis 210 advances second electrode 112 through atrial myocardium 404 and central fibrous body 406 to position second electrically active region 214 in ventricular myocardium 106 as shown in FIG. 4.

As second electrode 112 advances into the tissue, the distance between first electrode 114 and atrial endocardium 402 until first electrode 114 contacts, and may press against, the surface of atrial endocardium 402 so that heart tissue becomes engaged with first electrically active region 216. First electrode 114 is held in contact with atrial endocardium 402 by second electrode 112, e.g., retraction of first electrode 114 from the surface of atrial endocardium 402 is prevented by second electrode 112.

Target implant region 102 in some pacing applications is along atrial endocardium 402, substantially inferior to the AV node and bundle of His. Second electrode 112 can have a length that penetrates through atrial endocardium 402 in target implant region 102, through the central fibrous body 406, and into ventricular myocardium 106 without perforating through the ventricular endocardial surface. In some examples, when the full length of second electrode 112 is fully advanced into target implant region 102, second electrically active region 214 rests within ventricular myocardium 106 and first electrode 114 is positioned in intimate contact with atrial endocardium 402. Second electrode 112 may extend from housing distal end 202 approximately 3 millimeter (mm) to 12 mm in various examples. In some examples, second electrode 112 may extend a distance from housing 200 of at least 3 mm, at least 3 mm but less than 20 mm, less than 15 mm, less than 10 mm, or less than 8 mm in various examples. The diameter of first and second electrodes 114 and 112 may be less than 2 mm and may be less than or equal to 1 mm, or even less of equal to 0.6 mm.

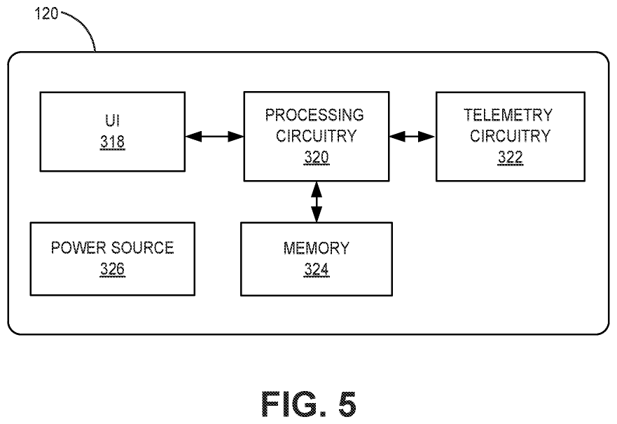

FIG. 5 is a block diagram illustrating an example configuration of computing device 120. Computing device 120 may include an user interface (UI) 318, processing circuitry 320, telemetry circuitry 322, memory 324, and power source 326. User interface 318 may transmit information and/or retrieve data and instructions from a user, such as a patient or clinician. In some examples, user interface 318 may transmit to the user an indication that cross-chamber capture (e.g., capture of a second chamber of heart 100) has been identified by device 104. In other examples, user interface 318 may receive instructions from users for dealing with the identified cross-chamber capture and transmit the instructions to processing circuitry 320.

Processing circuitry 320 may identify the presence of cross-chamber capture based on data transmitted from device 104 (e.g., from telemetry circuitry 312 of device 104 to telemetry circuit 322 of computing device 120). Processing circuitry 320 may also determine program parameters (e.g., cardiac pacing parameters, sensing parameters) that control the operation of device 104. In some examples, processing circuitry 322 may determine a response to the identification of the presence of cross-chamber capture, and instruct telemetry circuit 322 to transmit instructions to device 104 to carry out the response. For example, processing circuitry 320 may determine that the response to the identification of cross-chamber capture is to switch device 104 from a dual-chamber mode to a single-chamber mode. Processing circuitry 320 may then instruct telemetry circuit 322 to transmit instructions to switch to a single-chamber mode to device 104, e.g., through telemetry circuit 312 of device 104.

Memory 324 may store data received by computing device 120 from a user and/or device 104. In some examples, Memory 324 may store instructions for processing circuitry 322 to perform to identify and/or respond to the presence of cross-chamber capture. Power source 326 provides power to the other components of computing device 120. Power source 326 may be rechargeable and/or removable.

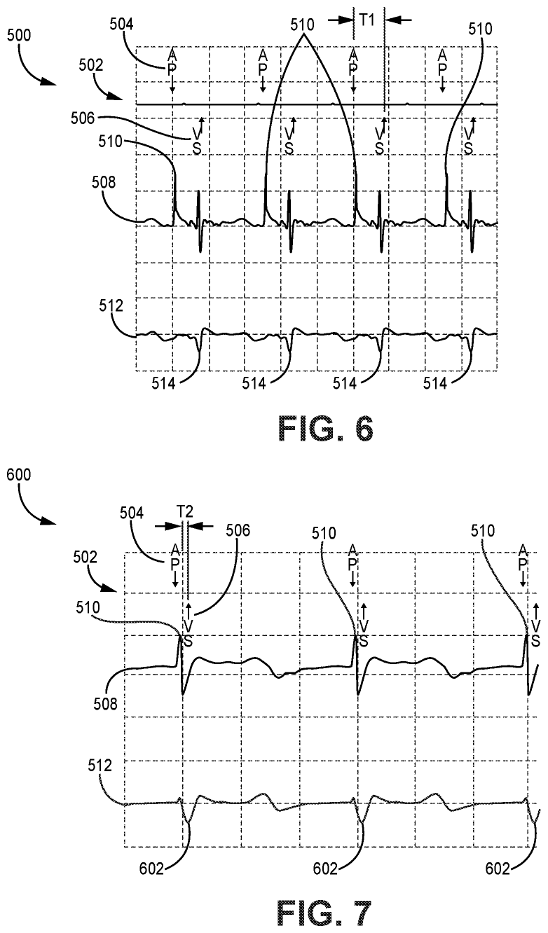

FIGS. 6 and 7 are plot diagrams showing example cardiac electrogram (EGM) graphs of the activity of a first chamber of heart 100 and second chamber of heart 100 under different circumstances. While the graphs of FIGS. 6 and 7 are directed to a RA as a first chamber of heart 100 and LV as a second chamber of heart 100, this disclosure may be directed to other combinations of chambers of heart 100.

FIG. 6 is a plot diagram showing an example EGM graph 500 of atrial pacing without causing ventricular capture. EGM graph 500 illustrates a first chamber electrical signal 508, the corresponding second chamber electrical signal 512, and a marker channel 502 for a set time period. Marker channel 502 is configured to annotate events in first chamber electrical signal 508 and second chamber electrical signal 512. The annotations may include atrial event markers 504 and ventricular event markers 506. Atrial event markers 504 includes a label (e.g., AP indicating atrial pace) that indicates that a pacing pulse was delivered to the RA resulting in pacing polarization artifact and evoked P-wave 510 visible on first chamber electrical signal 508. Ventricular event markers 506 indicate ventricular events, e.g., in the LV, and include a label (e.g., VS indicating ventricular sense). Marker channel 502 displays a time interval T1 between atrial event markers 504 and ventricular event markers 506. In some examples, as illustrated in FIG. 6, first chamber electrical signal 508 may correlate to electrical signals within the RA which are sensed by device 104, e.g., through sensing circuitry 304, first electrode 114 (e.g., in first electrically active region 216), and electrode 208. In some examples, as illustrated in FIG. 6, second chamber electrical signal 512 may correlate to electrical signals within the LV which are sensed by device 104, e.g., through sensing circuitry 304, second electrode 112 (e.g., in second electrically active region 214), and electrode 208.

First chamber electrical signal 508 may illustrate the electrical signals of the first chamber (e.g., the RA) in response to the delivery of stimulation signals through device 104. As illustrated in FIG. 6, first chamber electrical signal 508 may represent one or more P-waves 510. P-waves represent the depolarization of the RA in response to a stimulation signal from device 104. Second chamber electrical signal 512 illustrates the electrical signals corresponding to the response of the second chamber (e.g., the LV) to the depolarization of the RA. As illustrated in FIG. 6, second chamber electrical signal 512 may represent the Q-wave, R-wave, and S-wave (collectively “QRS complex 514”) of LV and RV. QRS complex 514 represents the depolarization of the LV. As illustrated in FIG. 6, time interval T1 is around 150 milliseconds (ms). In other examples, time interval T1 may be between 110 ms and 250 ms, depending upon the physiology of the patient, the health of heart 100, and stimulation signal parameters. Stimulation signal parameters may include the frequency of signal delivery and the pulse width and pulse amplitude of the stimulation signals. In some examples where time interval T1 is below a certain threshold (e.g., around 40 ms) or if there is a sudden change in the patient's average time interval, computing device 120 may determine that the length of time interval T1 may be attributed solely to non-physiological causes and that there is capture of ventricular tissue as a result of atrial the stimulation signals. In the example illustrated in FIG. 6, the length of time interval is around 150 ms. Therefore, computing device 120 may determine that there is no capture of ventricular tissue.

FIG. 7 is a plot diagram showing an example EGM graph 600 of the capture of LV in response to the delivery of stimulation signals to RA. First chamber electrical signal 508 illustrates P-waves 510 corresponding to the depolarization of RA in response device 104 delivering stimulation signals into wall tissue of RA, e.g., through first electrode 114. Second chamber electrical signal 512 illustrates a QRS complex 602 response to the stimulation signal. In the example illustrated in FIG. 7, Time interval T2 is 40 ms which is below the threshold, and computing device 120 will determine the presence of the capture of the LV.

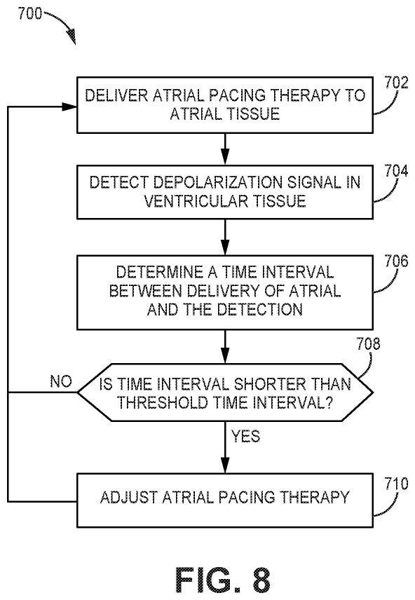

FIG. 8 is a flowchart illustrating an example method 700 for detecting the capture of a second chamber in response to the delivery of stimulation signals to a first chamber. Example method 700 may be performed using an implantable medical device (e.g., device 104 as shown in FIGS. 1-4). While example method 700 involves the RA as a first chamber of heart 100 and the LV as the second chamber of heart 100, other example methods may involve different chambers of heart 100. In other examples, the first and second chambers of heart 100 may be other combinations of the atria and/or ventricles. For example, the first chamber may be the LA and the second chamber may be the RV, in which case example method 700 may involve detecting the capture of the second chamber (RV), also referred to as cross-chamber capture, in response to the delivery of stimulation signals to the first chamber (LA). In some examples, the methods discussed in this disclosure may include additional or fewer steps than example method 700. In other examples, example method 700 may be performed using an implantable medical device with a different design than FIG. 104. For example, the implantable medical device may be implanted outside heart 100 with one or more leads extending from outside heart 100 into the RA. In other examples, implantable medical device may be configured to deliver stimulation signals to both a first and second chamber but also configured to detect the presence of cross-chamber capture in a third and/or fourth chamber. For example, a device may be configured to deliver stimulation signals to the LA and/or RA and be configured to detect the presence of capture in the RV and/or LV, respectively. Further, while described as being performed by device 104, e.g., processing circuitry 310 of device 104, the example technique of FIG. 8 may be performed, at least in part, by processing circuitry of another device, such as processing circuitry 320 of computing device 120 that wirelessly communicates with device 104 via telemetry circuitry 322.

Device 104 delivers an atrial pacing therapy to atrial tissue (702). Atrial pacing therapy may include one or more stimulation signals configured to induce depolarization of a first chamber (e.g., RA) at a predetermined pace. The parameters of the atrial pacing therapy may be determined through an external programmer and/or external computing device connected to device 104, e.g., through telemetry circuitry 312. In some examples, the parameters of the atrial pacing therapy may be stored in memory 314 of device 104. In some examples, the parameters of the atrial pacing therapy may be stored in memory 324 of computing device 120. In some examples, processing circuitry 310 of device 104 may be configured to determine an appropriate atrial pacing therapy for the RA of heart 100. Device 104 may deliver the atrial pacing therapy to atrial tissue through first electrode 114, which is in contact with the wall tissue of the RA.

Device 104 then detects a signal, e.g., an activation or depolarization signal, in the ventricular tissue of the RV (704) and determines a time interval between the delivery of the atrial pacing therapy and the detection of the signal in the ventricular tissue (706). Device 104 may detect a signal in the ventricular tissue of the LV (704) through sensing circuitry 304 and second electrode 112. The signal may be similar to second chamber electrical signal 508 as illustrated in FIG. 7 and may represent the QRS complex 508 of the LV. The time interval between the delivery of the atrial pacing therapy and the detection of the signal in the ventricular tissue may represent a PR interval, which may represent a time interval between the start of the P-wave in the RA and the start of the QRS complex in the LV. In some examples, processing circuitry 310 of device 104 may be configured to determine the time interval between the delivery of the atrial pacing therapy and the detection of the signal in the ventricular tissue.

Device 104 also determines if the time interval is shorter than (or less than or equal to) the threshold time interval (708). In some examples, processing circuitry 310 of device 104 may determine if the time interval is shorter than the threshold time interval. The threshold time interval may be a measurement of the time between an atrial pacing therapy and a signal in the ventricular tissue that may indicate the presence of ventricular capture. In some examples, the threshold time interval may be 110 ms or less or 80 ms or less. The threshold time interval may represent a short time interval where there is a high probability that the length of the time interval is a result of ventricular capture and not the physiology of the patient (e.g., health of heart 100, changes to health of heart 100). In some examples, threshold time interval may be based on previously recorded time intervals of the patient. For example, the threshold time interval may be one-half of the median time interval for the previous week or month. Processing circuitry 310 may determine the threshold time interval using sensing data stored in memory 314 of device 104, an external programmer, a computing device (e.g., computing device 120), a remote server, and/or the cloud.

Processing circuitry 310 may be configured to automatically determine if the time is shorter than the threshold time interval (708) on a regular basis. Processing circuitry 310 may make the determination on an hourly and/or daily basis. In some examples, processing circuitry 310 may make the determination as a part of one or more other regular tests performed on device 104. In some examples, processing circuitry 310 may be configured to make the determination based on instructions from the external programmer and/or the computing device.

If the time interval is not shorter than the threshold time interval (“NO” branch of 708), device 104 resumes delivering atrial pacing therapy to atrial tissue (702). If the time interval is shorter than the threshold time interval (“YES” branch of 708), then device 104 has identified the presence of the capture of the second chamber (e.g., the ventricular tissue of the LV, herein referred to as “ventricular capture”) as a result of the atrial pacing therapy and may adjust the atrial pacing therapy, e.g., through processing circuitry 310 of device 104. In some examples, device 104 may need to determine that a threshold number of time intervals shorter than the threshold time interval has been satisfied before determining that device 104 has identified the presence of the capture of the second chamber (i.e., cross-chamber capture). The threshold number of time intervals may be a number of short time intervals within a set number of detected interval. For example, device 104 may identify the presence of the capture of the second chamber upon a determination that six of the prior ten detected time intervals were shorter than the threshold time interval. In other examples, the threshold number may be the number of consecutive time intervals that are shorter than the threshold time interval.

In some examples, device 104 may adjust the atrial pacing therapy based on instructions from an external programmer or a computing device (e.g., computing device 120). In some examples, processing circuitry 310 may adjust the atrial pacing therapy by disabling delivering stimulation signals to the RA. Processing circuitry 310 may disable delivering stimulation signals to the RA temporarily or permanently. If processing circuitry 310 disables the delivery of stimulation signals temporarily, processing circuitry 310 may re-enable delivery of stimulation signals after the expiration of a time period. In other examples, processing circuitry 310 may re-enable the delivery of stimulation signals upon determining that there is no longer ventricular capture. In some examples processing circuitry 310 may permanently disable the delivery of stimulation signals until processing circuitry 310 receives instructions, from computing device 120 and through telemetry circuitry 312, to re-enable the delivery of stimulation signals to the RA.

In some examples, processing circuitry 310 may adjust the atrial pacing therapy by adjusting one or more parameters of the atrial pacing therapy. Processing circuitry 310 may adjust the parameters of the atrial pacing therapy by reducing the frequency, pulse width, and/or pulse amplitude of the stimulation signals. In some examples, processing circuitry 310 may configure the maximum adapted atrial amplitude or maximum adapted pulse width within an atrial capture management feature of device 104. For example, processing circuitry 310 may set the maximum values to which device 104 may adjust these values in response to loss of atrial capture. In some examples, processing circuitry 310 may switch device 104 from a dual-chamber mode (e.g., a dual-chamber pacing mode) to a single-chamber mode (e.g., a single-chamber synchronous pacing mode (VDD)). Processing circuitry 310 may switch device 104 from the single-chamber mode to the dual-chamber mode after the expiration of a certain time period or in response to a determination that there is no longer ventricular capture. In some examples, processing circuitry 310 may switch device 104 from the single-chamber mode to the dual-chamber mode in response to instructions from computing device 120.

In some examples, processing circuitry 310 may confirm that ventricular capture is occurring in a confirmation step prior to adjusting the atrial pacing therapy. Upon a further confirmation of ventricular capture in the confirmation step, processing circuitry 310 may then adjust the atrial pacing therapy. If processing circuitry 310 does not confirm ventricular capture, device 104 may return to detecting signals in the ventricular tissue (704). While example method 800 involves the RA as a first chamber of heart 100 and the LV as the second chamber of heart 100, other example methods may involve different chambers of heart 100.

In some examples, processing circuitry 310 may also send a notification to the external programmer and/or computing device 120, e.g., through telemetry circuitry 312 of device 104, that processing circuitry 310 has identified the presence of ventricular capture. In some examples, processing circuitry 310 may send a notification to one or more mobile computing devices such as a laptop or a mobile phone.

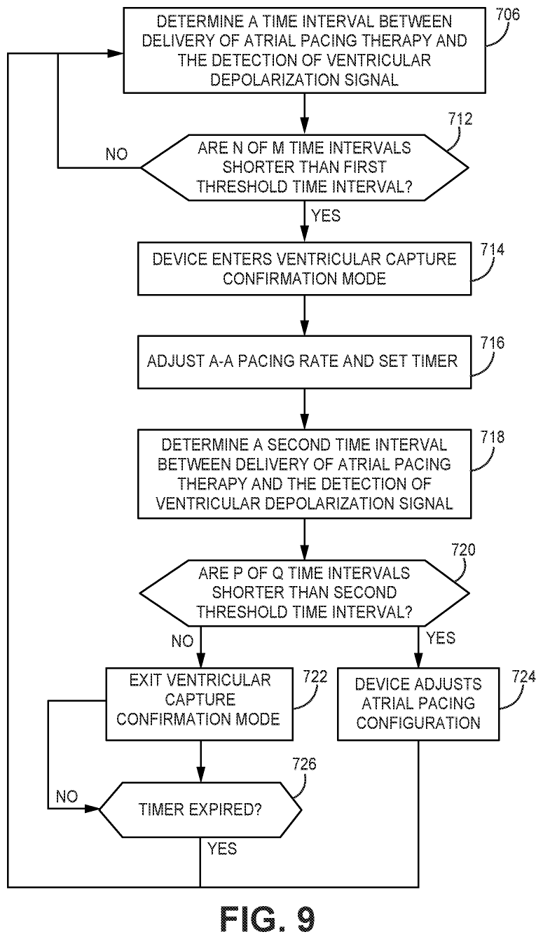

FIG. 9 is a flowchart illustrating an example method for confirming the detection of the capture of a second chamber in response to the delivery of stimulation signals to a first chamber. While described as being performed by device 104, e.g., processing circuitry 310 of device 104, the example method of FIG. 9 may be performed, at least in part, by processing circuitry of another device, such as processing circuitry 320 of computing device 120.

Device 104 determines a time interval between the delivery of atrial pacing therapy and the detection of the signal (706), e.g., in accordance with the examples disclosed herein with respect to FIG. 8. Device 104 then determines if device 104 has detected a threshold number of time intervals (e.g., N of M intervals) that are shorter than the first threshold time interval (712).

If device 104 does not detect a threshold number of time intervals that are shorter than a first threshold time interval, e.g., if less than N of M time intervals are shorter than the first threshold time interval (“NO” branch of 712), device 104 resumes determining a time interval between the delivery of atrial pacing therapy and the detection of the signal (706). In some examples, device 104 may also stop detecting depolarization signals in ventricular tissue for a set period of time (e.g., until the termination of a timer) as a result of a determination that device 104 did not detect the threshold number of time intervals (“NO” branch of 712). If device 104 does detect the threshold number of time intervals that are shorter than the first threshold time interval, e.g., if device 104 detects at least N of M time intervals are shorter than the first threshold time interval (“YES” branch of 712), then device 104 enters a ventricular capture confirmation mode (714).

While device 104 is in the ventricular capture confirmation mode, device 104 may continue detecting depolarization signals in ventricular tissue and determine the corresponding time intervals. Device 104 may adjust A-A pacing rate of the pacing therapy and set a timer (716). A-A pacing rate may include the frequency at which device 104 outputs stimulation signals, e.g., the number of stimulation signals (e.g., atrial pacing signals) device 104 outputs for a given time period. Device 104 may adjust A-A pacing rate by increasing the frequency of stimulation signal output or shortening a time interval between successive stimulation signals.

Device 104 may determine a second time interval between the delivery of atrial pacing therapy and the detection of ventricular depolarization signal (718), e.g., in accordance with examples discussed herein. If device 104, while in the ventricular capture confirmation mode, does not detect a second threshold number of time intervals that are shorter than a second threshold time interval, e.g., if less than P of Q time intervals are shorter than the second threshold time interval (“NO” branch of 720), device 104 exits the ventricular capture confirmation mode (722). While device 104 is not in the ventricular capture confirmation mode, device 104 may determine if a timer has expired (726). If device 104 determines that a timer has expired (“YES” branch of 726), device 104 may determine the time interval between delivery of atrial pacing therapy and the detection of ventricular depolarization signal (706) and restart the process. If device 104 determines that the timer has not expired (“NO” branch of 726), device 104 must wait (e.g., at step 722 as illustrated in FIG. 9) until device 104 determines that the timer has expired (726). In some examples, the timer may be determined based on user-input through UI 318 of computing device 120 or may be determined by device 104 and/or computing device 120 based on prior data.

If device 104 does detect the second threshold number of time intervals, e.g., if device 104 detects at least P of Q time intervals that are shorter that the second threshold time interval (“YES” branch of 720), then device 104 adjusts the atrial pacing configuration (724). In some examples, the second threshold number of time intervals may be satisfied by a single time interval that is shorter than the second threshold time interval.

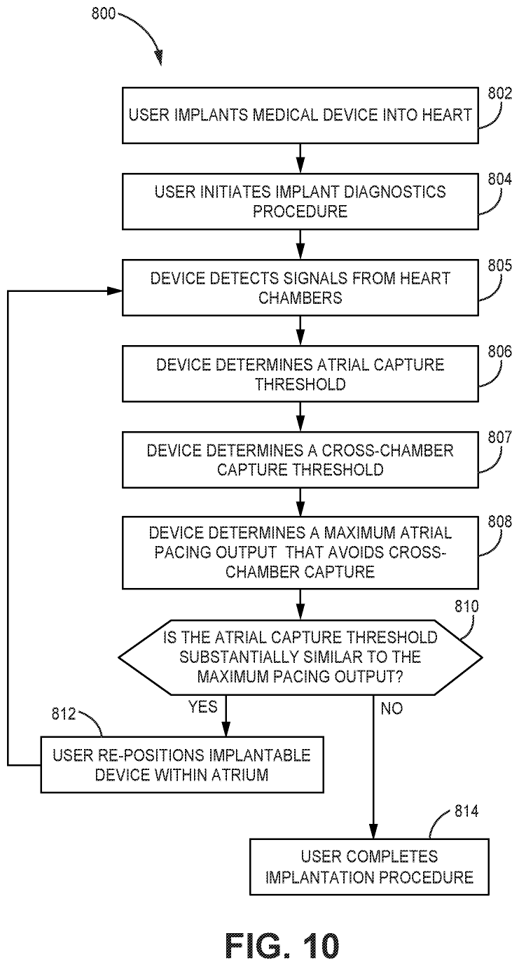

FIG. 10 is a flowchart illustrating an example method 800 for detecting the capture of a second chamber in response to the delivery of stimulation signals to a first chamber during the implantation of an implantable medical device (e.g., device 104). While described as being performed by device 104, e.g., processing circuitry 310 of device 104, the example technique of FIG. 10 may be performed, at least in part, by processing circuitry of another device, such as processing circuitry 320 of computing device 120. In some examples, the example technique of FIG. 10 may be performed for the implantation of one or more implantable medical leads of a medical device.

To begin example method 800, a user (e.g., a physician, a clinician) first implants device 104 into heart 100 of a patient (802) and initiates implant diagnostics procedures (804). As part of implant diagnostics procedures, processing circuitry 310 may instruct signal generation circuitry 306 and switch circuitry 304 of device 104 to deliver test stimulation pulses into the atrial wall tissue of the RA. Device 104 also detects signals from the first and second heart chambers (e.g., RA and the LV, respectively) in response to the test stimulation pulses (805). Device 104 may determine whether the stimulation pulses captured the first or second chambers based on the signals. Device 104 may detect the signals using sensing circuitry 304, sensor(s) 308, and electrodes 112, 114, and 208. The electrical signals may be similar to the first chamber electrical signals 602 and second chamber electrical signals 604 as illustrated in FIG. 7. Test stimulation pulses may be pulses with different frequencies, pulse widths, and pulse amplitudes. Device 104 may deliver test stimulation pulses into the atrial wall tissue of the RA to determine a suitable combination of parameter settings for the patient. Device 104 may also determine an atrial capture threshold (806) for heart 100. Atrial capture threshold may be a combination of pacing settings that captures the atrium and may include a range of frequencies, pulse widths, and pulse amplitudes for the stimulation signals delivered by device 104. In some examples, processing circuitry 310 may determine the atrial capture threshold based on detecting capture of the atrium via electrode 114 and sensing circuitry 304.

Device 104 may determine a cross-chamber capture threshold (807) and may determine a maximum atrial pacing output before ventricular capture occurs (808). The cross-chamber capture threshold may be the minimum parameters of an atrial stimulation signal that may trigger a ventricular capture and may be defined by the frequency, pulse width, and pulse amplitude of the atrial stimulation signal. The maximum atrial pacing output may be the maximum parameters of an atrial stimulation signal that does not satisfy the cross-chamber capture threshold. In some examples, an atrial stimulation signal with a higher frequency, larger pulse width, and/or higher pulse amplitude than the maximum atrial pacing output will trigger ventricular capture in heart 100. In some examples, processing circuitry 310 of device 104 may determine the cross-chamber capture threshold and the maximum atrial pacing output.

Device 104, or another device, such as computing device 120, or a user, may also determine whether the atrial capture threshold is substantially similar to the maximum pacing output (810). If the atrial capture threshold is substantially similar to the maximum atrial pacing output, delivery of atrial pacing has a substantial likelihood of triggering ventricular capture and/or may not be maintained safely without triggering cross-chamber capture. In some examples, processing circuitry 310 determines whether the atrial capture threshold is substantially similar to the maximum pacing output (810). Substantial similarity may mean that there is less than a threshold difference between the atrial capture threshold and the maximum atrial pacing output. In some examples, the threshold difference may be a safety margin value and the atrial capture threshold may be substantially similar to the maximum pacing output if the net voltage of the sum of the atrial capture threshold and the safety margin value is equal to or exceeds the cross-chamber capture threshold. In some examples, the safety margin value may be between about 0.5 V and about 1.5 V. In some examples, the threshold difference may be a safety margin factor and the atrial capture threshold may be substantially similar to the maximum pacing output if the net voltage of the atrial capture threshold multiplied by the safety margin factor is equal to or exceeds the cross-chamber capture threshold. In some examples, the safety margin factor may be between about 1.5 and about 2.5.

If device 104, computing device 120, or the user determines that the atrial capture threshold is not substantially similar to the maximum pacing output (“NO” branch of 810), the user may complete the implantation procedure (814). In some examples, processing circuitry 310 may send a notification to the external programmer and/or computing device that there is not a risk of cross-chamber capture by device 104. In some examples, processing circuitry 310 may be configured to, as part of an atrial pacing threshold testing, to automatically increase the atrial pacing output as an atrial capture threshold of device 104 increases. Processing circuitry 310 may be further configured, as part of completing the implantation procedure (814), to determine a maximum adapted amplitude for atrial pacing such that potential outputs of device 104 do not exceed the maximum atrial pacing output, e.g., the threshold at which (or determined based on the threshold at which) atrial pacing results in ventricular capture.

If device 104, computing device 120, or the user determines that the atrial capture threshold is substantially similar to the maximum pacing output (“YES” branch of 810), e.g., through processing circuitry 310 of device 104, then the user may re-position device 104 within the RA (812). In some examples, user re-positions device 104 within the RA such that first electrode 114 and second electrode 112 is secured at a location within target area 102. In some examples, processing circuitry 310 may send a notification to the external programmer and/or computing device, e.g., via a network, that device 104 needs to be repositioned within the RA. In some examples, processing circuitry 310 may send a notification to the external programmer and/or computing device to reposition device from a first position within the RA to a second position within the RA.

Once user has re-positioned device 104 within the RA (812), device 104 reinitiates implant diagnostics procedures (804) in accordance with the procedure discussed above. In some examples, device 104 may notify user that device 104 needs to be repositioned after device 104, computing device 120, or a user determines that the atrial capture threshold is substantially similar to the maximum atrial pacing output. If device 104 determines that a certain number of the selected atrial pacing therapies are substantially similar to the maximum pacing output, device 104 may then notify user that device 104 needs to be re-positioned.

It should be understood that various aspects disclosed herein may be combined in different combinations than the combinations specifically presented in the description and accompanying drawings. It should be understood that, depending on the example, certain acts or events of any of the processes or methods described herein may be performed in a different sequence, may be added, merged, or left out altogether (e.g., all described acts or events may not be necessary to carry out the techniques. In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

In addition, it should be noted that system described herein may not be limited to treatment of a human patient. In alternative examples, the system may be implemented in non-human patient, e.g., primates, canines, equines, pigs, and felines. These other animals may undergo clinical or research therapies that may benefit from the subject matter of this disclosure.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Example 1: a device comprising: a housing; a first electrode and a second electrode extending from the housing, wherein the first electrode contacts tissue of a first chamber of a heart, and wherein the second electrode is configured to penetrate into tissue of a second chamber of the heart that is separate from the first chamber of the heart; sensing circuitry within the housing wherein the sensing circuitry is configured to sense electrical activity of the first chamber via the first electrode and electrical activity of the second chamber via the second electrode; therapy delivery circuitry within the housing, wherein the therapy delivery circuitry is configured to deliver cardiac pacing to the first chamber via the first electrode and the second chamber via the second electrode; and processing circuitry within the housing, wherein the processing circuitry is configured to: identify, based on the sense electrical activity of the second chamber, a capture of the tissue of the second chamber in response to delivery of cardiac pacing to the first chamber by the therapy delivery circuitry; and adjust the cardiac pacing to the first chamber based on the identification of the capture of the second chamber.

Example 2: the device of example 1, wherein, to identify the capture of the second chamber in response to delivery of cardiac pacing to the first chamber by the therapy delivery circuitry via the first electrode, the processing circuitry is configured to: determine a time interval to a signal sensed by the sensing circuitry via the second electrode; compare the time interval to a threshold time interval; and identify the capture based on the comparison.

Example 3: the device of example 2, wherein the time interval comprises a time interval from the delivery of the cardiac pacing via the first electrode to the signal sense via the second electrode.

Example 4: the device of examples 2 or 3, wherein the processing circuitry is configured to determine the threshold time interval using one or more previously recorded time intervals.

Example 5: the device of any of examples 2-4, wherein the processing circuitry is configured to identify the capture of the tissue of the second chamber based on delivery of cardiac pacing to the first chamber by identifying that an amount of pacing pulses delivered to the first chamber that capture the second chamber satisfies a threshold.

Example 6: the device of any of examples 2-5 wherein the processing circuitry is configured to confirm the capture of the tissue of the second chamber prior to adjusting the delivery of cardiac pacing.

Example 7: The device of example 6, wherein the time interval comprises a first time interval, and to confirm the capture of the tissue of the second chamber, the processing circuitry is configured to: compare one or more second time intervals from delivery of cardiac pacing to the first chamber to a signal sensed in the second chamber to a second threshold time interval, wherein the second threshold time interval is shorter than the threshold time interval; and determine that a threshold number of the one or more second time intervals are shorter than the second threshold time interval.

Example 8: the device of any of examples 1-7, wherein the second chamber is a ventricle.

Example 9: the device of any of examples 1-8, wherein the first chamber is an atrium.

Example 10: the device of any of examples 1-9, wherein to adjust the cardiac pacing to the first chamber, the processing circuitry is configured to adjust the delivery of the cardiac pacing.

Example 11: the device of example 10, wherein the processing circuitry is configured to adjust the delivery of the cardiac pacing by disabling delivery of the cardiac pacing to the first chamber in response to the identification of the capture of the tissue of the second chamber.

Example 12: the device of example 11, wherein the processing circuitry is further configured to re-enable the delivery of the cardiac pacing to the first chamber in response to at least one expiration of a time period or a user command.

Example 13: the device of any of examples 10-12, wherein the processing circuitry is further configured to adjust the delivery of the cardiac pacing by switching the device from a dual-chamber pacing mode to a single-chamber pacing mode in response to the identification of the capture of the tissue of the second chamber.

Example 14: the device of any of examples 10-13, wherein the processing circuitry is configured to adjust the delivery of the cardiac pacing by maintaining the device in a dual-chamber sensing mode in response to the identification of the capture of the tissue of the second chamber.

Example 15: the device of any of examples 1-9, wherein to adjust the cardiac pacing to the first chamber, the processing circuitry is configured to adjust one or more settings that affect the cardiac pacing.

Example 16: the device of example 15, wherein the one or more settings that affect the cardiac pacing comprises a maximum amplitude for a pacing output of the cardiac pacing.

Example 17: The device of any of examples 1-9, wherein to adjust the cardiac pacing to the first chamber, the processing circuitry is configured to adjust pacing output of the cardiac pacing.

Example 18: the device of example 17, wherein to adjust the pacing output of the cardiac pacing, the processing circuitry is configured to control the therapy delivery circuitry to decrease a pulse magnitude of the cardiac pacing.

Example 19: the device of any of examples 17 and 18, wherein to adjust the pacing output of the cardiac pacing, the processing circuitry is configured to control the therapy delivery circuitry to decrease a pulse width of the cardiac pacing.

Example 20: the device of any of examples 1-19, wherein the processing circuitry is configured to identify the capture of the tissue of the second chamber in response to a command from a user.

Example 21: the device of any of examples 1-20, further comprising communication circuitry within the housing, wherein the processing circuitry is configured to send a notification to an external device via the communication circuitry in response to the identification of the capture of the tissue of the second chamber.

Example 22: The device of any of examples 1-21, wherein the device is wholly implantable within the first chamber of the heart.

Example 23: the device of any of examples 1-22, wherein the first electrode and the second electrode are electrically coupled to a lead, and wherein the lead is configured to extend outside the heart to the housing.

Example 24: the device of any of examples 1-23, wherein the device further comprises a return electrode, and wherein the sensing circuitry is further configured to sense electrical activity of the first chamber and electrical activity of the second chamber via the return electrode.

Example 25: the device of example 24, wherein the therapy delivery circuitry is further configured to deliver cardiac pacing to the first chamber and the second chamber via the return electrode.

Example 26: a method comprising: delivering cardiac pacing from a device to a first chamber of a heart via a first electrode of the device; sensing, by the device and via a second electrode of the device that penetrates into tissue of a second chamber of the heart that is separate from the first chamber of the heart, electrical activity of the second chamber; identifying, by the device and based on the sensed electrical activity of the second chamber, a capture of the tissue of the second chamber in response to delivery of cardiac pacing to the first chamber; and adjusting, by the device, the cardiac pacing to the first chamber by the therapy delivery circuitry in response to the identification of the capture of the second chamber.

Example 27: the method of example 26, wherein identifying the capture of the tissue of the second chamber in response to delivery of cardiac pacing to the first chamber via the first electrode comprises: determining a time interval to a signal sensed by the sensing circuitry via the second electrode; comparing the time interval to a threshold time interval; and identifying the capture based on the comparison.

Example 28: the method of example 27, wherein the time interval comprises a time interval from the delivery of the cardiac pacing via the first electrode to the signal sensed via the second electrode.

Example 29: the method of any of examples 27 and 28, further comprising determining the threshold time interval using one or more previously recorded time intervals.

Example 30: the method of any of examples 27-29, wherein identifying the capture of the tissue of the second chamber comprises identifying an amount of pacing pulses delivered to the first chamber that captures the second chamber that satisfy a threshold.

Example 31: the method of any of examples 27-30, wherein confirming the capture of the tissue of the second chamber comprises confirming the capture of the tissue based on delivery of cardiac pacing to the first chamber prior to adjusting the delivery of cardiac pacing.

Example 32: the method of example 31, wherein the time interval comprises a first time interval, and wherein confirming the capture of the tissue of the second chamber comprises: comparing one or more second time intervals from delivery of cardiac pacing to the first chamber to a signal sensed in the second chamber to a second threshold time interval, wherein the second threshold time interval is shorter than the threshold time interval; and determining that a threshold number of the one or more second time intervals are shorter than the second threshold time interval.

Example 33: the method of any of examples 26-32, wherein the second chamber is a ventricle.

Example 34: the method of any of examples 26-33, wherein the first chamber is an atrium.

Example 35: the method of any of examples 26-34, wherein adjusting the cardiac pacing to the first chamber comprises adjusting, by the device, the delivery of the cardiac pacing.

Example 36: the method of example 35, wherein adjusting the delivery of the cardiac pacing comprises disabling the delivery of cardiac pacing to the first chamber in response to the identification of the capture of the tissue of the second chamber.

Example 37: the method of example 36, wherein adjusting the delivery of the cardiac further comprises re-enabling delivery of cardiac pacing to the first chamber in response to at least one expiration of a time period or a user command.

Example 38: the method of any of examples 35-37, wherein adjusting the delivery of cardiac further comprises switching the device from a dual-chamber pacing mode to a single-chamber pacing mode in response to the identification of the capture of the tissue of the second chamber.

Example 39: the method of any of examples 35-38 wherein adjusting the delivery of the cardiac pacing further comprises maintaining the device in a dual-chamber sensing mode in response to the identification of the capture of the tissue of the second chamber.

Example 40: the device of any of examples 26-34, wherein adjusting the cardiac pacing to the first chamber comprises adjusting, by the device, one or more settings that affect the cardiac pacing.

Example 41: the method of example 40, wherein the one or more settings that affect the cardiac pacing comprises a maximum amplitude for a pacing output of the cardiac pacing.

Example 42: the method of any of examples 26-34, wherein adjusting the cardiac pacing to the first chamber comprises adjusting, by the device, pacing output of the cardiac pacing.

Example 43: the method of example 42, wherein adjusting the pacing output of the cardiac pacing comprises decreasing a pulse magnitude of the cardiac pacing.

Example 44: the method of any of examples 42 and 43, wherein adjusting the pacing output of the cardiac pacing comprises decreasing a pulse width of the cardiac pacing.

Example 45: the method of any of examples 26-44, further comprising identifying the capture of the tissue of the second chamber in response to a command from a user.

Example 46: the method of any of examples 25-43, further comprising sending a notification, by the device, to an external device in response to the identification of the capture of the tissue of the second chamber.

Example 47: a method comprising: delivering test cardiac pacing pulses from a device to a first chamber of a heart via a first electrode of the device; detecting, by the device and via the first electrode and a second electrode of the device that penetrates into tissue of a second chamber of the heart that is separate from the first chamber of the heart, electrical activity of the first chamber and the second chamber, respectively; determining, by the device and based on the sensed electrical activity of the first chamber, a capture threshold of the tissue of the first chamber; determining, by the device and based on the sensed electrical activity of the second chamber, a cross-chamber capture threshold of the tissue of the second chamber and a maximum pacing output for the first chamber; and based on a comparison of the capture threshold and the maximum pacing output, communicating, by the device to another device via a network, an indication to adjust a position of the first electrode within the first chamber.

Example 48: the method of example 47, wherein the maximum pacing output comprises a maximum cardiac pacing delivered by the device to the first chamber that does not capture the tissue of the second chamber, and wherein the method further comprises: determining that there is less than a threshold difference between the atrial capture threshold and the maximum pacing output; and communicating the indication based on the determination.

Example 49: the method of any of examples 47 and 48, wherein the cross chamber capture threshold of the tissue of the second chamber comprises a minimum cardiac pacing delivered by the device to the first chamber that captures the tissue of the second chamber, and wherein determining the maximum pacing output comprises determining, by the device, the maximum pacing output based on the cross-chamber capture threshold.

Example 50: the method of any of examples 47-49, wherein the second chamber is a ventricle.

Example 51: the method of any of examples 47-50, wherein the first chamber is an atrium.

Example 52: the method of any of examples 47-51, wherein the indication to adjust the position of the first electrode comprises an indication to re-position the first electrode from a first position within the first chamber to a second position within the first chamber.

Example 53: a computer readable storage medium comprising instructions that, when executed, cause processing circuitry within a device to perform the method of any of claims 26-46.

Example 54: a computer readable storage medium comprising instructions that, when executed, cause processing circuitry within a device to perform the method of any of examples 47-52.

1. A device comprising:
a housing;
a first electrode and a second electrode, wherein the first electrode is configured to contact tissue of a first chamber of a heart, and wherein the second electrode is configured to penetrate into tissue of a second chamber of the heart that is separate from the first chamber of the heart;
sensing circuitry within the housing, wherein the sensing circuitry is configured to sense electrical activity of the first chamber via the first electrode and electrical activity of the second chamber via the second electrode;
therapy delivery circuitry within the housing, wherein the therapy delivery circuitry is configured to deliver cardiac pacing to the first chamber via the first electrode and the second chamber via the second electrode; and
processing circuitry within the housing, wherein the processing circuitry is configured to:
identify, based on the sensed electrical activity of the second chamber, a capture of the tissue of the second chamber in response to delivery of cardiac pacing to the first chamber by the therapy delivery circuitry via the first electrode; and
adjust the cardiac pacing to the first chamber based on the identification of the capture of the second chamber.

2. The device of claim 1, wherein, to identify the capture of tissue of the second chamber in response to delivery of cardiac pacing to the first chamber by the therapy delivery circuitry via the first electrode, the processing circuitry is configured to:
determine a time interval to a signal sensed by the sensing circuitry via the second electrode;
compare the time interval to a threshold time interval; and
identify the capture based on the comparison.

3. The device of claim 2, wherein the time interval comprises a time interval from the delivery of the cardiac pacing via the first electrode to the signal sensed via the second electrode.

4. The device of claim 2, wherein the processing circuitry is configured to determine the threshold time interval using one or more previously recorded time intervals.

5. The device of claim 2, wherein the processing circuitry is configured to identify the capture of the tissue of the second chamber based on delivery of cardiac pacing to the first chamber by identifying that an amount of pacing pulses delivered to the first chamber that capture the second chamber satisfies a threshold.

6. The device of claim 2, wherein the processing circuitry is configured to confirm the capture of the tissue of the second chamber based on delivery of cardiac pacing to the first chamber prior to adjusting the delivery of cardiac pacing.

7.-9. (canceled)

10. The device of claim 1, wherein to adjust the cardiac pacing to the first chamber, the processing circuitry is configured to adjust the delivery of the cardiac pacing by disabling delivery of the cardiac pacing to the first chamber in response to the identification of the capture of the tissue of the second chamber.

11. The device of claim 10, wherein the processing circuitry is further configured to re-enable the delivery of the cardiac pacing to the first chamber in response to at least one expiration of a time period or a user command.

12. The device of claim 1, wherein to adjust the cardiac pacing to the first chamber, the processing circuitry is configured to adjust the delivery of the cardiac pacing by switching the device from a dual-chamber pacing mode to a single-chamber pacing mode in response to the identification of the capture of the tissue of the second chamber.

13. The device of claim 1, wherein to adjust the cardiac pacing to the first chamber, the processing circuitry is configured to adjust the delivery of the cardiac pacing by maintaining the device in a dual-chamber sensing mode in response to the identification of the capture of the tissue of the second chamber.

14.-15. (canceled)

16. A method comprising:
delivering cardiac pacing from a device to a first chamber of a heart via a first electrode of the device;
sensing, by the device and via a second electrode of the device that penetrates into tissue of a second chamber of the heart that is separate from the first chamber of the heart, electrical activity of the second chamber;
identifying, by the device and based on the sensed electrical activity of the second chamber, a capture of the tissue of the second chamber in response to delivery of cardiac pacing to the first chamber; and
adjusting, by the device, the cardiac pacing to the first chamber by the therapy delivery circuitry in response to the identification of the capture of the second chamber.

17. The method of claim 16, wherein identifying the capture of the tissue of the second chamber in response to delivery of cardiac pacing to the first chamber via the first electrode comprises:
determining a time interval to a signal sensed by the sensing circuitry via the second electrode;
comparing the time interval to a threshold time interval; and
identifying the capture based on the comparison.

18. The method of claim 16, wherein adjusting the cardiac pacing to the first chamber comprises adjusting, by the device, the delivery of the cardiac pacing by disabling the delivery of cardiac pacing to the first chamber in response to the identification of the capture of the tissue of the second chamber.

19. The method of claim 18, wherein adjusting the delivery of the cardiac further comprises re-enabling, by the device, the delivery of cardiac pacing to the first chamber in response to at least one expiration of a time period or a user command.

20. The method of claim 16, wherein adjusting the cardiac pacing to the first chamber comprises adjusting, by the device, the delivery of the cardiac pacing by switching the device from a dual-chamber pacing mode to a single-chamber pacing mode in response to the identification of the capture of the tissue of the second chamber.

21. The method of claim 16, wherein adjusting the cardiac pacing to the first chamber comprises adjusting, by the device, the delivery of the cardiac pacing by maintaining the device in a dual-chamber sensing mode in response to the identification of the capture of the tissue of the second chamber.

22. A method comprising:
delivering test cardiac pacing pulses from a device to a first chamber of a heart via a first electrode of the device;
detecting, by the device and via the first electrode and a second electrode of the device that penetrates into tissue of a second chamber of the heart that is separate from the first chamber of the heart, electrical activity of the first chamber and the second chamber, respectively;
determining, by the device and based on the sensed electrical activity of the first chamber, a capture threshold of the tissue of the first chamber;
determining, by the device and based on the sensed electrical activity of the second chamber, a cross-chamber capture threshold of the tissue of the second chamber and a maximum pacing output for the first chamber; and
based on a comparison of the capture threshold and the maximum pacing output, communicating an indication to adjust a position of the first electrode within the first chamber.

23. The method of claim 22, wherein the maximum pacing output comprises a maximum cardiac pacing delivered by the device to the first chamber that does not capture the tissue of the second chamber, and wherein the method further comprises:
determining that there is less than a threshold difference between the capture threshold and the maximum pacing output; and
communicating the indication based on the determination.

24. The method of claim 22, wherein the cross-chamber capture threshold of the tissue of the second chamber comprises a minimum cardiac pacing output delivered by the device to the first chamber that captures the tissue of the second chamber, and wherein determining the maximum pacing output comprises determining, by the device, the maximum pacing output based on the cross-chamber capture threshold.

25. The method of claim 22, wherein the indication to adjust the position of the first electrode comprises an indication to re-position the first electrode from a first position within the first chamber to a second position within the first chamber.
